(19) United States
(12) Patent Application Publication
(10) Pub. No.: US 2026/0363787 A1
(43) Pub. Date: Nov. 19, 2026

(54) Simultaneous Multi-View Live Targeting Leveraging Patient-Mounted Reference Bodies

(71) Applicant: Stryker European Operations Limited, Carrigtwohill (IE)

(72) Inventors: Jakob Kemper, Heemstede (NL); Ebenezer Peters, Eden Prairie, MN (US); Ulrich Hoffmann, Breisach (DE); Fabian Huegle, March (DE)

(21) Appl. No.: 19/135,619

(22) PCT Filed: Dec. 13, 2022
(86) PCT No.: PCT/EP2022/085677

§ 371 Date: Jun. 4, 2025

(51) Int. Cl. A61B 34/20; A61B 90/00; A61B 90/50; A61B 90/92

(52) U.S. Cl. CPC A61B 34/20; A61B 90/92; A61B 2034/2055; A61B 2034/2065; A61B 2090/376; A61B 2090/3937; A61B 2090/3966; A61B 2090/3995; A61B 2090/502

(57) ABSTRACT

Surgical tracking system allowing an improved localization of surgical components, in particular a simultaneous multi-view live targeting of reference bodies, and a corresponding method.

### FIELD OF THE INVENTION

The present invention relates to a surgical tracking system, and in particular to a surgical tracking system allowing an improved localization of surgical components, in particular a simultaneous multi-view live targeting of reference bodies, and a corresponding method, computer program product and storage medium having stored thereon the computer program product.

### BACKGROUND OF THE INVENTION

Surgical procedures have improved over the recent years. Significant improvements have been achieved by supporting systems for supporting the clinical personal, in particular surgeons during surgeries. In particular, bone fractures benefit from supporting systems for surgeons, which provide the surgeon with equipment, which allows the surgeon to improve exactness of repositioning of bone parts and positioning of implants, like screws, nails and bone plates, as well as tools and targeting and guiding devices.

As traumatized bones, i.e., fractures, have only a limited visual access, monitoring is usually based on radiating principles, like X-ray imaging or computer tomography CT images, or magnet resonance tomography MRT images. All these principles and methods involve at least one of the drawbacks of being radiation intensive, requiring large devices and requiring a considerable amount of time. Each monitoring step during a surgery prolongs the surgery duration and thus the duration of narcotic impact and increases costs and radiation impact.

Therefore, there is a need for surgical tracking systems as well as a corresponding method, which allow an improved targeting based on reference bodies, which reduces imaging effort and thus duration of the surgery, reduces radiation impact on the patient, but at the same time maintain or increase the level of exactness of the surgery.

### SUMMARY OF THE INVENTION

The present invention provides a surgical tracking system, a surgical tracking method and a surgical navigation system according to the subject matter of the independent claims. Further embodiments are incorporated in the dependent claims.

The present invention provides a surgical tracking system for tracking a plurality of surgical inventories with respect to each other, the surgical tracking system comprises a first optical pattern representing a position and orientation of a first surgical inventory, the first optical pattern having at least one first unique optical sub-pattern; a second optical pattern representing a position and orientation of a second surgical inventory, the second optical pattern having at least one second unique optical sub-pattern; a surgeon worn device comprising a first optical imaging device and an optical display device; and an image-processing device comprising a pattern recognition means being adapted for recognizing the position and orientation of the first sub-pattern of the first optical pattern and the position and orientation of second sub-pattern of the second optical pattern based on an image collected by said first optical imaging device and a stored representation of the first optical pattern and the second optical pattern, a position-determining means being adapted for determining a relative position and orientation of the first optical pattern and the second optical pattern with respect to a position and viewing direction of the first optical imaging device based on the position and orientation of the first sub-pattern of the first optical pattern and the position and orientation of second sub-pattern of the second optical pattern recognized by said pattern recognition means and visualization means being adapted for virtually visualizing said first surgical inventory represented by the first optical pattern and said second surgical inventory represented by the second optical pattern as seen from a surgeon wearing said surgeon worn device and providing said visualizing to said optical display device.

Thus, a device may be provided which allows real time displaying for a surgeon a situation of a surgery, as seen from a surgeon, including positions and orientations of components, which cannot be recognized by a surgeon in a real view. Even hidden objects can be displayed at any time, even if the objects have moved and no further fluoroscopic image had been taken. The recognized optical patterns allow determination of the position and orientation of said patterns. As a position and orientation of an object with respect to the respective optical pattern is known, also the position and orientation of the objects represented by said optical patterns can be determined. The virtual representation of the surgery inventory allows displaying a situation virtually by only tracking the optical patterns The situation can be displayed for a surgeon at the same time of image taking of the optical patterns. As both, the optical imaging device and the optical display device is provided at the surgeon worn device, the true perspective may be maintained for the surgeon.

According to an embodiment, the surgical tracking system further comprising a second optical imaging device to be positioned within the environment of a surgical procedure, wherein said pattern recognition means being adapted for recognizing the position and orientation of the first sub-pattern of the first optical pattern and the position and orientation of the second sub-pattern of the second optical pattern based on an image collected by said second optical imaging device and a stored representation of the first optical pattern and the second optical pattern; wherein the position-determining means being adapted for determining a relative position and orientation of the surgeon worn device and the second optical imaging device with respect to the first optical imaging device, and for determining a relative position and orientation of the first optical pattern and the second optical pattern with respect to a position and viewing direction of the first optical imaging device based on an image taken from the second optical imaging device, the determined relative position and orientation of the surgeon worn device and the second optical imaging device with respect to the first optical imaging device and a stored representation of the first optical pattern and the second optical pattern.

Thus, a redundant imaging can be carried out by a second optical imaging device, which allows an independent determination of the relative position and orientation of the surgeon worn imaging device with respect to the first and second optical pattern.

According to an embodiment the image-processing device is adapted for identifying whether for at least one of the first optical pattern and the second optical pattern image no respective sub-pattern is recognizable upon optical imaging by one of the first optical imaging device and the second optical imaging device, wherein the position-determining means being adapted for determining a relative position and orientation of the first optical pattern and the second optical pattern with respect to a position and viewing direction of the first optical imaging device based on imaging by the other of the first optical imaging device and the second optical imaging device.

Thus, the surgical tracking system allows taking over the pattern recognition and determination of the position and orientation based on one of the optical imaging devices, in case the optical patterns are obscured for the other of the optical imaging devices. In case the surgeon positions a tool between an optical pattern and the optical imaging device provided on the surgeon worn optical imaging device, the imaging can be taken over by the other imaging device, so that the displaying can be continued without interruption.

According to an embodiment, the image-processing device further comprises an augmenting means being adapted for augmenting at least one of a further surgical inventory and an operating trajectory of a surgical inventory onto the virtual visualization of the first surgical inventory and the second surgical inventory based on a recognized position and orientation of the first optical sub-pattern of the first optical pattern and the second optical sub-pattern of the second optical pattern with respect to a position and viewing direction of the first optical imaging device, so as to visualize said further surgical inventory and said operating trajectory of a surgical inventory, respectively, relative to said first surgical inventory represented by the first optical pattern and said second surgical inventory represented by the second optical pattern.

Thus, it is possible to visualize for the surgeon during surgery both, real objects and virtual objects. Real objects may be objects being visible for the surgeon from the present viewpoint. Virtual objects may be objects being not visible for a surgeon from the present viewpoint. The reason therefor may be that the objects are hidden, e.g., by the patient's anatomy, as they are already introduced into the patient's body, or that the objects are actually not in position, but visualized, so that the surgeon may recognize whether the intended position or the kind of object, e.g., its size fits the expectations.

According to an embodiment, the image-processing device further comprises at least one of a first surgical inventory and a second surgical inventory, wherein the respective first optical pattern and second optical pattern is inseparable mounted to the corresponding respective one of the at least first surgical inventory and second surgical inventory, wherein the respective optical pattern representing a position and orientation of the respective surgical inventory.

Thus, the respective surgical inventory can be made part of the system. With this respect, the respective optical pattern may be fixedly mounted to the respective surgical inventory or may be releasably mounted to the surgical inventory.

According to an embodiment, at least one of a first surgical inventory and a second surgical inventory is at least one of a surgical tool/instrument, surgical implant, a surgical reference body, a patient's anatomy, and a fluoroscopic imaging device, wherein the at least one of the first surgical inventory and the second surgical inventory comprises a radio dense geometry having a unique radio projection for each proximal to distal orientation of the respective one of the first surgical inventory and the second surgical inventory, and being fixedly and spatially reproducibly connected to the respective one of the first surgical inventory and the second surgical inventory.

Thus, a position and orientation of the surgical inventory having fixed thereto a radio dense reference body may also be determined based on fluoroscopic imaging. This allows comparison of both determinations of a relative position and orientation, and a verification of the position and orientation of said surgical inventory. This may also be used for calibration of the system at the beginning, but also once or repeatedly during surgery. Also deviations may be detected and computationally be corrected. It should be understood, that optical imaging usually will be carried out more often than fluoroscopic imaging, as the amount of x-ray radiation should be kept as low as possible.

According to an embodiment, at least one of the first optical pattern and the second optical pattern comprises a mechanical interface to be coupled to a positive fit receptacle of a surgical inventory for forming a unit having a reproducible positional relation between a geometry of said surgical inventory, and the respective optical pattern.

Thus, a reproducible determination of the relative position and orientation of two or more surgical inventories or objects may be carried out based on the determination of the relative position and orientation of the respective optical patterns or sub-patterns.

According to an embodiment, the optical pattern is a three-dimensional optical pattern.

Thus, a three-dimensional optical pattern can be combined with a radio dense geometry including a plurality of spatially three dimensionally distributed fiducial markers. Further, a three-dimensional optical pattern may provide a number of optical sub-patterns which have a characteristic difference for different viewing directions. This may allow an identification of a rough viewing direction based on the identified optical sub pattern. A further specification of the viewing direction may be achieved when providing a polygonal body having a characteristic sub-pattern on each side, so that based on those sub-patterns being visible from a particular viewing direction a more specific determination of the viewing direction may be determined. The polygon may be cube with different characteristic sub-patterns on each of the six sides of a cube. Also other polygons may be used upon need.

According to an embodiment the optical pattern is composed of a characteristic bar or block code along sides of a square.

Thus, the square may serve as a general reference and the relation of the bar or block code may be identified easily.

According to an embodiment the optical pattern is composed of a geometrically even raster of fields of different colors, in particular a raster of squared colored fields, in particular a raster of color gradient fields.

Thus, not only light and dark fields can be used, but also different colors. This allows a color coding which makes it easier for a surgeon to select the right optical pattern. Further when using instead of two options more than two colors, i.e., light and dark, then more information can be stored on the same surface portion. With two options, e.g., light and dark, two fields can reflect four different combinations. With four options, e.g., yellow, blue, red, green, two fields can reflect sixteen different combinations, which is four times more.

According to an embodiment the surgical instrument is a surgical guiding device and further comprises a guiding body having a longitudinal extension from a proximal end of the surgical guiding device to a distal end of the surgical guiding device, and being adapted for guiding at least one of a longitudinal surgical implant and a longitudinal tool, and having a guiding trajectory extending along the guiding body and succeeding in distal direction along a traveling path of at least one of a surgical implant and a surgical tool to be inserted and guided; and a radio dense geometry being located in a predetermined spatial position and orientation with respect to the guiding body, and being adapted for providing a unique radio projection for each proximal to distal orientation of the guiding body.

Thus, the surgical guiding device may be monitored by optical imaging based on the optical patterns and fluoroscopic imaging based on the radio dense geometry.

According to an embodiment the surgical reference body comprises a radio dense geometry being fixedly and spatially reproducibly connected to the surgical reference body, a reference body portion having a mechanical interface for being connected to a patient's anatomy, wherein the radio dense geometry has a unique radio projection for each proximal to distal orientation of the surgical reference body, so that the radio dense geometry allows determination of the spatial position and orientation of the surgical reference body based on a two dimensional radio projection of at least a part of the surgical reference body.

Thus, it is possible to not only determine the relative position and orientation based on an optical pattern recognition and identification, but at the same time monitoring the surgical reference body, be it in form of an integrally formed reference body or an attachable reference body, also by radio monitoring. Thus, both identification options can be provided, which also may be used at the same time.

According to an embodiment the surgical reference body comprises a radio dense geometry having a first radio dense sub-geometry and a second radio dense sub-geometry each being fixedly and spatially reproducibly connected to the surgical reference body; a first reference body portion having a mechanical interface for being connected to a patient's anatomy; and a second reference body portion having a mechanical interface for being connected to a patient's anatomy, wherein each of the first radio dense sub-geometry and the second radio dense sub-geometry has a unique radio projection for each proximal to distal orientation of the surgical reference body, so that each of the first radio dense sub-geometry and the second radio dense sub-geometry alone allows determination of the spatial position and orientation of the surgical reference body based on a two dimensional radio projection of at least a part of the surgical reference body, wherein the first radio dense sub-geometry is allocated to the first reference body portion, and the second radio dense sub-geometry is allocated to the second reference body portion.

Thus, it is possible to reduce the focus on the relevant portion of the reference body, in particular when using a larger reference body. Depending on the viewing direction upon radio imaging, only parts of the entire reference body may be seen in a radio image. Therefore, it is relevant that position and orientation can be determined even if having only a partial view. If providing a plurality of radio dense sub-geometry, each allowing the determination of the spatial position and orientation, it is very likely that at least one of the radio dense sub-geometries is within the imaged portion, and thus allows determination of the position and orientation of the reference body.

According to an embodiment, there is provided a surgical tracking method for tracking a plurality of surgical inventories with respect to each other, the surgical tracking method comprises: optical imaging by a first optical imaging device on a surgeon worn device of a surgical environment including a first optical pattern representing a position and orientation of a first surgical inventory and a second optical pattern representing a position and orientation of a second surgical inventory; recognizing a position and orientation of a first sub-pattern of the first optical pattern and the position and orientation of a second sub-pattern of the second optical pattern based on an image collected by said first optical imaging device and a stored representation of the first optical pattern and the second optical pattern; determining a relative position and orientation of the first optical pattern and the second optical pattern with respect to a position and viewing direction of the first optical imaging device based on the position and orientation of the recognized first sub-pattern of the first optical pattern and the position and orientation of the recognized second sub-pattern of the second optical pattern; visualizing said first surgical inventory represented by the first optical pattern and said second surgical inventory represented by the second optical pattern as seen from a surgeon; and providing said visualizing to an optical display device on a surgeon worn device.

Thus, a method may be provided which allows real time visualization for a surgeon a situation of a surgery, as seen from a surgeon, including positions and orientations of components, which cannot be recognized by a surgeon in a real view. Even hidden objects can be displayed at any time, even if the objects have moved and no further fluoroscopic image had been taken. The recognized optical patterns allow determination of the position and orientation of said patterns. As a position and orientation of an object with respect to the respective optical pattern is known, also the position and orientation of the objects represented by said optical patterns can be determined. The virtual representation of the surgery inventory allows displaying a situation virtually by only tracking the optical patterns. The situation can be displayed for a surgeon at the same time of image taking of the optical patterns. As both, the optical imaging device and the optical display device is provided at the surgeon worn device, the true perspective may be maintained for the surgeon.

According to an embodiment the surgical tracking method further comprises optical imaging by a second optical imaging device positioned within the environment of a surgical procedure and apart from the first optical imaging device a surgical environment including a first optical pattern representing a position and orientation of a first surgical inventory and a second optical pattern representing a position and orientation of a second surgical inventory, recognizing a position and orientation of a first sub-pattern of the first optical pattern and the position and orientation of a second sub-pattern of the second optical pattern based on an image collected by said second optical imaging device and a stored representation of the first optical pattern and the second optical pattern; determining a relative position and orientation of the second optical imaging device with respect to the first optical imaging device; determining a relative position and orientation of the first optical pattern and the second optical pattern with respect to a position and viewing direction of the first optical imaging device based on an image taken from the second optical imaging device, the determined relative position and orientation of the surgeon worn device and the second optical imaging device with respect to the first optical imaging device and a stored representation of the first optical pattern and the second optical pattern.

Thus, a redundant imaging can be carried out by a second optical imaging device, which allows an independent determination of the relative position and orientation of the surgeon worn imaging device with respect to the first and second optical pattern.

According to an embodiment the surgical tracking method further comprises identifying whether for at least one of the first optical pattern and the second optical pattern image no respective sub-pattern is recognizable upon optical imaging by one of the first optical imaging device and the second optical imaging device, determining a relative position and orientation of the first optical pattern and the second optical pattern with respect to a position and viewing direction of the first optical imaging device based on optical imaging by the other of the first optical imaging device and the second optical imaging device.

Thus, the surgical tracking method allows taking over the pattern recognition and determination of the position and orientation based on one of the optical imaging devices, in case the optical patterns are obscured for the other of the optical imaging devices. In case the surgeon positions a tool between an optical pattern and the optical imaging device provided on the surgeon worn optical imaging device, the imaging can be taken over by the other imaging device, so that the displaying can be continued without interruption.

According to an embodiment the surgical tracking method further comprises augmenting at least one of a further surgical inventory and an operating trajectory of a surgical inventory onto the virtual visualization of the first surgical inventory and the second surgical inventory based on a recognized position and orientation of the first optical sub-pattern of the first optical pattern and the second optical sub-pattern of the second optical pattern with respect to a position and viewing direction of the first optical imaging device, visualizing said further surgical inventory and said operating trajectory of a surgical inventory, respectively, relative to said first surgical inventory represented by the first optical pattern and said second surgical inventory represented by the second optical pattern.

Thus, it is possible to visualize for the surgeon during surgery both, real objects and virtual objects. Real objects may be objects being visible for the surgeon from the present viewpoint. Virtual objects may be objects being not visible for a surgeon from the present viewpoint. The reason therefor may be that the objects are hidden, e.g., by the patient's anatomy, as they are already introduced into the patient's body, or that the objects are actually not in position, but visualized, so that the surgeon may recognize whether the intended position or the kind of object, e.g., its size fits the expectations.

According to an embodiment the surgical tracking method further comprises fluoroscopic imaging of a surgical environment including said first surgical inventory and said second surgical inventory, each having fixedly and spatially reproducibly connected thereto a radio dense marker having a unique radio projection for each proximal to distal orientation of said surgical inventory; determining a position and orientation of each of said surgical inventories based on the fluoroscopic imaging and a stored spatial relation of each of the surgical inventories and a respective radio dense geometry with respect to each other; establishing coincidence between a position and orientation of each of said surgical inventories determined based on the fluoroscopic imaging of respective radio dense markers and a position and orientation of each of said surgical inventories determined based on the optical imaging of respective optical markers.

Thus, the position and orientation of the surgical inventory can be verified. The coincidence may also be used for calibration at the beginning ore during surgery. The calibration may be carried out once or repeatedly, e.g., after having finished a crucial surgical process and/or before starting a crucial surgical process.

According to an embodiment, a surgical navigation method is provided for navigating a plurality of surgical inventories with respect to each other, the surgical navigation method comprises (A) fluoroscopic imaging of a surgical environment including said plurality of surgical inventories, each having fixedly and spatially reproducibly connected thereto a radio dense marker having a unique radio projection for each proximal to distal orientation of said surgical inventory; (B) determining a position and orientation of each of said plurality of surgical inventories based on the fluoroscopic imaging and a stored spatial relation of each of the surgical inventories and a respective radio dense geometry with respect to each other; (C) optical imaging by a first optical imaging device on a surgeon worn device a surgical environment including a first optical pattern representing a position and orientation of a first surgical inventory and a second optical pattern representing a position and orientation of a second surgical inventory, (D) recognizing a position and orientation of a first sub-pattern of the first optical pattern and the position and orientation of a second sub-pattern of the second optical pattern based on an image collected by said first optical imaging device and a stored representation of the first optical pattern and the second optical pattern; (E) determining a relative position and orientation of the first optical pattern and the second optical pattern with respect to a position and viewing direction of the first optical imaging device based on the position and orientation of the recognized first sub-pattern of the first optical pattern and the position and orientation of the recognized second sub-pattern (90a) of the second optical pattern; (F) establishing coincidence between a position and orientation of each of said plurality of surgical inventories determined based on the fluoroscopic imaging of respective radio dense markers and a position and orientation of each of said plurality of surgical inventories determined based on the optical imaging of respective optical markers; (G) visualizing said first surgical inventory represented by the first optical pattern and said second surgical inventory represented by the second optical pattern (90) as seen from a surgeon; (H) providing said visualizing to an optical display device on a surgeon worn device; and (I) iteratively repeating steps (A), (B), (C), (D), (E), (G), and (H) for navigating a plurality of surgical inventories based on optical imaging.

Thus, the surgical tracking method may be used for permanent navigation and updating the respective procedural steps and the display of the surgeon worn device may be updated iteratively.

According to an embodiment, there is provided a surgical method comprising the steps of optical imaging using a first optical imaging device on a surgeon worn device a surgical environment including a first optical pattern representing a position and orientation of a first surgical inventory and a second optical pattern representing a position and orientation of a second surgical inventory, determining a position and orientation of a first sub-pattern of the first optical pattern and the position and orientation of a second sub-pattern of the second optical pattern based on an image collected by said first optical imaging device and a stored representation of the first optical pattern and the second optical pattern; determining a relative position and orientation of the first optical pattern and the second optical pattern with respect to a position and viewing direction of the first optical imaging device based on the position and orientation of the recognized first sub-pattern of the first optical pattern and the position and orientation of the recognized second sub-pattern of the second optical pattern, and placing said first surgical inventory represented by the first optical pattern with reference to said second surgical inventory represented by the second optical pattern as seen from a surgeon based on visualizing said first surgical inventory and second surgical inventor on an optical display device of said surgeon worn device.

Thus, a surgical method can be provided, which may be conducted assisted by the aforementioned and described surgical tracking system.

According to an embodiment of the invention, there is provided a computer program product, which when carried out executes the method as describe above.

According to an embodiment of the invention, there is provided a data storage medium having stored thereon an executable code of the computer program product as described above.

It should be noted that the above described embodiments may also be combined and in a combined form provide a synergetic technical effect and synergetic benefits which go beyond the sum of the single technical effects and benefits.

### BRIEF DESCRIPTION OF THE FIGURES

The invention will be described by way of the following drawings, wherein

FIG. 1: illustrates an exemplary embodiment of a surgical guiding device/surgical instrument/tool in a lateral view.

FIG. 2: illustrates an exemplary embodiment of a surgical guiding device/surgical instrument/tool in a perspective view seen from proximal to distal direction.

FIG. 3: illustrates a schematic view of a principal structure and set-up of an exemplary embodiment of the surgical tracking system.

FIG. 4: illustrates a side view in a radio image of a surgical guiding device/surgical instrument/tool applied to a patient's anatomy when using the surgical tracking system.

FIG. 5: illustrates a schematic view of a complimentary match of a first and second radio dense sub-geometry.

FIG. 6 illustrates a perspective view of a situation with the application of an embodiment of the surgical tracking system to surgical inventories, where an optical imaging device is provided at a surgeon worn device.

FIG. 7 illustrates a perspective view of a situation with the application of a further exemplary embodiment of the surgical tracking system to surgical inventories, where a further external optical imaging device is provided.

FIG. 8 illustrates a perspective view of a situation with the application of a further exemplary embodiment of the surgical tracking system to surgical inventories, where an anatomy is represented by a surgical reference body.

FIG. 9 illustrates a perspective view of a situation with the application of a further exemplary embodiment of the surgical tracking system to surgical inventories, where also an instrument is provided with a representing optical pattern.

FIG. 10: illustrates a schematic view of an exemplary embodiment where the optical imaging device is connected to the surgeon worn device via respective interfaces and the pattern is connected to the reference body connected via respective interfaces.

FIG. 11: illustrates a schematic view of an exemplary embodiment where the first optical pattern is connected to an instrument via respective interfaces and the second optical pattern is connected to the surgical reference body connected via respective interfaces.

FIG. 12: illustrates a schematic view of an exemplary embodiment of a method with mandatory and optional method steps.

It should be noted that same or similar reference numerals illustrate same or similar components. Along these Figures exemplary embodiments of the invention will be describes as follows.

### DETAILED DESCRIPTION OF EXEMPLARY EMBODIMENTS

For a surgical implant and a surgical tool, the distal end is defined as the end firstly entered into a patient's body, and the proximal end is defined as the opposite end. For a drilling tool, the end including the drilling geometry is considered as being the distal end and the shaft for fixing the drilling tool to a drilling drive is considered as being the proximal end.

A unique optical pattern is considered as an optical pattern, which characteristically differs from other optical patterns and allow identification of said pattern based on its unique character. A sub-pattern is considered as a part of an optical pattern. A unique sub-pattern usually also makes the optical pattern to which it belongs unique. A unique optical pattern may include more than one unique sub patterns. For the described invention, an optical sub pattern may also be considered as the entire optical pattern. In other words, an optical pattern or unique optical pattern may consist of only a sub pattern or unique sub pattern, but may also consist of additional pattern portions, which again may be unique sub patterns or even non-unique pattern portions.

A (radio) projection is considered as projected image of a geometry, in particular a three-dimensional geometry onto a two-dimensional array.

Complementary patterns of a first and second radio dense sub-geometry are considered as matching patterns, which together form a closed common pattern. Such matching patterns may be formed e.g., by concentric circles or polygons or other shapes having a uniform circumferential distance or overlap, by interleaving segments having a uniform distance or overlap like segments of a circle or polygons or other shapes, etc.

Centre line of a tool, an implant or a part thereof is the imaginary line, which follows a path, which has an equal distance to the lateral edges of the respective tool, implant or part thereof.

Complementary patterns of a first and second radio dense sub-geometry are considered as matching patterns, which together form a closed common pattern. Such matching patterns may be formed e.g., by concentric circles or polygons or other shapes having a uniform circumferential distance or overlap, by interleaving segments having a uniform distance or overlap like segments of a circle or polygons or other shapes, etc.

Virtually visualizing a surgical guiding device or a surgical instrument may include a full visualization of the surgical guiding device and surgical instrument, respectively, but in addition or alternatively may also include visualization of a characteristic geometry, which may be an axis of the surgical guiding device and surgical instrument, respectively, and/or a representative scale and/or contour thereof. Virtually visualizing a surgical guiding device or a surgical instrument may also include a visualization of an available variety of implants or the like, e.g., visualizing three different available bone screws, in particular in combination with the patient's anatomy to which a screw is intended to be applied, so that the surgeon may recognize and identify through the virtually visualization the suitable screw out of the variety of screws. It should be noted that this is not limited to the number of three and also not limited to screws, but may also include nails, in particular nails with varying curve radius and other implants and surgical instruments like k-wires and the like.

A unique projection of any intended use orientation of the surgical guiding device or a surgical instrument, does not exclude that a projection of two or more different orientations are identical, as long as the system and/or the surgeon recognizes that the second and each further orientation with identical projection are outside an intended or reasonable use. With this respect a repeated pattern projection may be acceptable, if it always guaranteed, that the orientation within an intended or reasonable use range can be determined based on the unique projection. Outside an intended or reasonable use may be seen if the surgical guiding device or a surgical instrument is upside down oriented or toward an orientation, which cannot lead to serious injuries during surgery.

The correspondence between a reference body and a patient's anatomy can be established by providing a plurality (two or more) images from different positions/orientations (e.g., ML, AP or any other different directions) with the reference body being attached to the patient's anatomy. Based on these plurality of views the relation between the reference body and the patient's anatomy is achieved by image augmentation. The known geometry of a reference body allows determining the scaling of the reference body and the instrument/tool/anatomy in the imaging plane. The different imaging views may be referenced with respect to each other. Further, an automated or manual 2D-image segmentation can be carried out for setting different reference bodies in relation to a patient's anatomy. This can be supported by a database, which includes generally known bone geometries or individually known bone geometries, which can be obtained by e.g., a postoperative CT or the like.

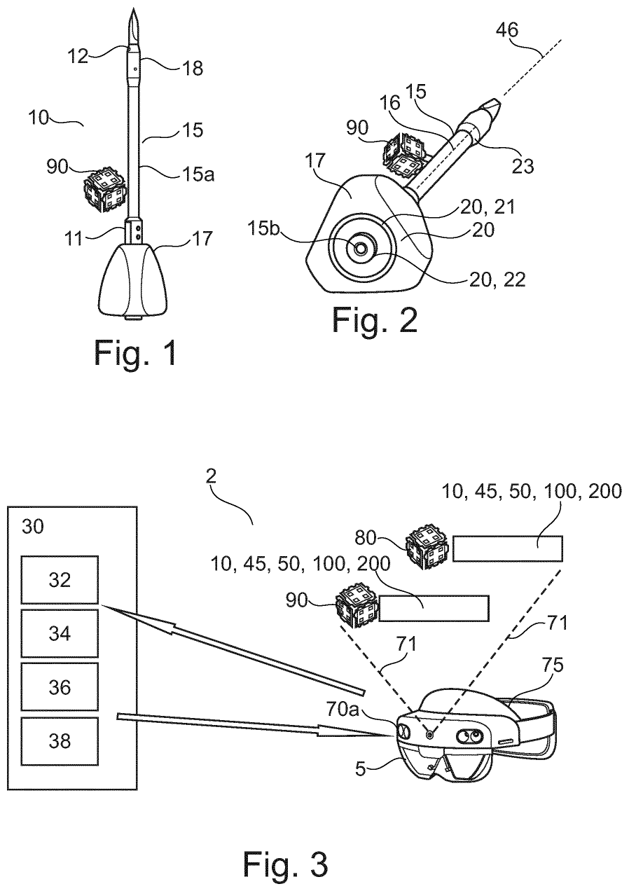

FIG. 1 and FIG. 2 illustrate for use in a surgical guiding system 1 for computer-assisted-surgery CAS a surgical guiding device 10, which is here illustrated as an awl. The surgical guiding device 10 comprises a guiding body 15 having a longitudinal extension from a proximal end 11 of the surgical guiding device to a distal end 12 of the surgical guiding device 10. The guiding body has a hollow shaft 15a, and being adapted for guiding at least one of a longitudinal surgical implant and a longitudinal tool. The surgical implant may be e.g. a screw or a nail or a wire. A tool may be a k-wire, a drill or a needle. The hollow shaft 15a has a guiding channel 15b, wherein the guiding channel follows the guiding trajectory 16 extending along the guiding body and succeeding in distal direction along a traveling path 46 of a surgical implant or a surgical tool to be inserted and guided. The guiding trajectory may be straight or may be bended or curved. A straight trajectory may be used for inserting straight implants or tools, like a drill or a screw. A bended trajectory may be used for inserting bended or curved implants or tools, like bended nails, or bended wires. The guiding trajectory is to be understood as the trajectory within or on the guiding body 15 or hollow shaft. The traveling path 46 is to be understood as a path extending the guiding path 16 toward the distal direction, i.e., the direction pointing toward the patient. The traveling path usually has a similar curvature as the guiding trajectory 16. If the guiding trajectory 16 is straight, also the traveling path 46 is straight. If the guiding path is curved, usually also the traveling path along which a curved implant or tool is traveling is curved. The traveling path defines the path the guided implant or tool when being inserted travels after exiting the hollow shaft 15a at the distal end, which is here illustrated as the tip 18 of the tool. Here, the illustrated awl has a handle or knob 17, which is used for handling the awl, in particular for applying a blade at the distal end 12 of the awl. The blade may leave an opening through which a tool or implant may be guided through the hollow shaft and through the distal opening toward the patient.

FIG. 2 illustrates at the knob 17 a radio dense geometry 20. The radio dense geometry 20 is located in a predetermined spatial position and orientation with respect to the guiding body 15. The radio dense geometry 20 provides a unique radio projection 25 for any proximal to distal orientation of the guiding body in an intended use orientation of the surgical guiding device 10. As the unique projection allows determining the orientation of the guiding device 10 and thus the guiding body 15 and the hollow shaft 15a, the unique projection can be used for determining the guiding trajectory 16 and the traveling path 46 along which a tool or implant travels when being guided by the hollow shaft 15a. It should be noted that the hollow shaft may also have a lateral slit (not illustrated here) for laterally inserting an implant or tool. This slit may be closed by a cover so as to for a closed hollow shaft 15a. The guiding body (15) comprises a hollow shaft (15a) with a guiding channel (15b), wherein the guiding channel follows the guiding trajectory (16). The radio dense geometry 20 may have a first radio dense sub-geometry 21 and a second radio dense sub-geometry 22. The first and second radio dense sub-geometries 21, 22 in this illustration are located at the knob 17 but may also be located somewhere on the guiding device 10. Any radio dense geometry 20 or sub-geometry 21, 22, 23 may also be provided as a releasable mounted geometry, e.g., with a clip connection. Sub-geometry 21, 22 may be formed together in a clip. Matching key-keyhole elements on the guiding device and the radio dense geometry 20 or a radio dense sub-geometry 21/22, 23 may establish a predefined orientation and position of the radio dense geometry 20/sub-geometry 21, 22, 23 with respect to the guiding device. The key/keyhole components may also be used that only radio dense geometries 20/sub-geometries 21, 22, 23 which are intended for being used with the guiding device 10 can be clipped to the guiding device. The radio dense geometry may have a unique three-dimensional shape and/or may be composed of sub-geometries together forming the unique projection. The first radio dense sub-geometry 21 and the second radio dense sub-geometry 22 may be realized by two circular rings of a radio opaque material, as illustrated in FIG. 2, which are concentrically arranged but not in the same plane but parallel planed. If having a view straight from the proximal to the distal direction, both rings in the projection appear as concentric circles. In this viewing direction both rings, one of the first radio dense sub-geometry 21 and one of the second radio dense sub-geometry 22 may form a complimentary pattern, here two concentric rings, which may fit into each other. If applying an inclined view from slightly lateral positon, the rings appear as ellipses and no longer concentric. The measure of the concentric shift and the measure of the elliptic deformation, as well as the relative size of both rings may give a basis for calculating not only the lateral viewing angle, but also the viewing distance. As the geometry of the guiding device is known, also its guiding trajectory 16 is knows and thus the traveling path 46. This applies not only for a straight guiding trajectory, but also to a curved guiding trajectory 16. The radio projection 26 of the first radio dense sub-geometry 21 and the radio projection 27 of the second radio dense sub-geometry 22 together in a predetermined viewing direction, which may be toward the straight longitudinal extension, may have a complementary pattern 29, as illustrated in FIG. 5. This complementary pattern 29 may be formed by e.g. the both concentric rings. Other complementary patterns may be formed by any key/keyhole shapes matching to each other when viewing toward the complementary viewing direction.

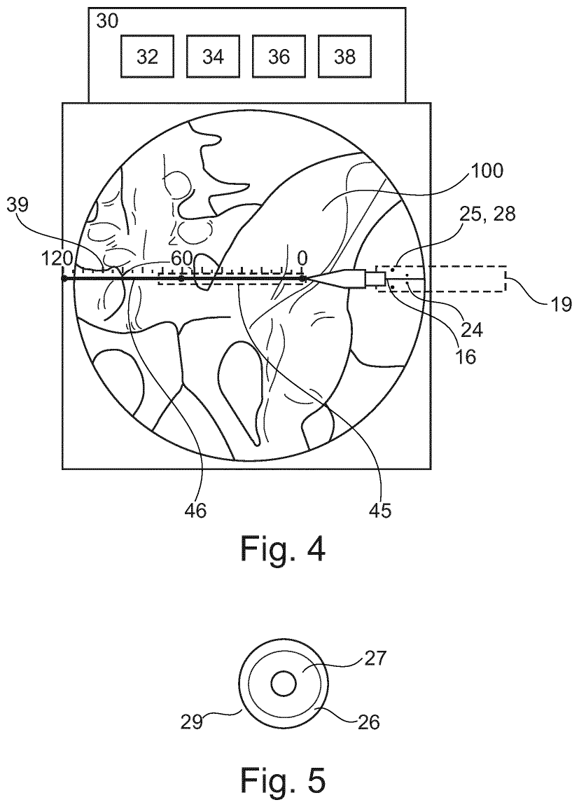

As illustrated in FIG. 2 the guiding device may have a further, a third radio dense sub-geometry 23. Whereas the first and second radio dense sub-geometries 21 and 22 in the shown embodiment are located at the proximal end 11 with the knob 17, the third radio dense sub-geometry 23 is located close to the distal end 12 and the tip 18. The third radio dense sub-geometry 23 may be provided with fiducial markers 24, as illustrated in FIG. 4. It should be noted that the third radio dense sub-geometry 23 may also be provided at the proximal end 11 and may also spatially overlap with the first and second radio dense sub-geometries 21 and 22. Fiducial markers 24 may be radio opaque spheres or other geometries, which are spatially arranged so as to commonly provide unique projection for any viewing direction. The concentric circles of the first and second radio dense sub-geometries 21 and 22 may allow a very exact determination of the exact proximal to distal direction, the fiducial markers 24 may allow an exact lateral determination of the spatial orientation. It should be understood that although FIGS. 1 and 2 illustrate an awl, the guiding device 10 may also be a targeting device for positioning a nail or a screw.

FIG. 3 illustrates a surgical tracking system for tracking a plurality of surgical inventories with respect to each other according to an exemplary embodiment. The surgical tracking system 2 comprises a first optical pattern 80 representing a position and orientation of a first surgical inventory 10, 45, 50, 100, 200. The first optical pattern 80 has at least one first unique optical sub-pattern 80a, which is here provided on one side of a cube. The surgical tracking system further comprises a second optical pattern 90 representing a position and orientation of a second surgical inventory 10, 45, 50, 100, 200, wherein the second optical pattern 90 also has at least one second unique optical sub-pattern 90a, which is here illustrated as a unique optical pattern on one side of a cube. The center portion of each pattern of each cube side may be identical and may serve for recognition of the presence of a pattern at all. The sides of the square here are provided with a unique block or bar code, which allows identification of that particular pattern. The optical sub patterns of different optical patterns may be unique in a way to allow to not only determination of the position and orientation of the optical pattern and the connected object, but also the identification of the connected object. A surgeon worn device 5 is used for visualizing the surgical situation by a surgeon. The surgeon worn device, e.g., an augmented reality goggle or VR glasses, e.g., a Hololens device, comprises a first optical imaging device 70a for imaging the optical patterns 80, 80a, 90, 90a and an optical display device 75. The surgical tracking system further comprises an image-processing device 30 having a pattern recognition means 32 being adapted for recognizing the position and orientation of the first optical pattern or the first sub-pattern 80a of the first optical pattern 80 and the position and orientation of the second optical pattern 90 or the second sub-pattern 90a of the second optical pattern 90 based on an image collected by said first optical imaging device 70a and a stored representation of the first optical pattern 80 and the second optical pattern 90. The stored representation of the optical pattern may be a stored image or a data set which allows identification thereof. The image-processing device 30 also comprises a position-determining means 34 being adapted for determining a relative position and orientation of the first optical pattern 80 and the second optical pattern 90 with respect to a position and viewing direction 71 of the first optical imaging device 70a based on the position and orientation of the first sub-pattern 80a of the first optical pattern 80 and the position and orientation of second sub-pattern 90a of the second optical pattern 90 recognized by said pattern recognition means 32. Then, the visualization means 36 virtually visualizes said first surgical inventory 10, 45, 50, 100, 200 represented by the first optical pattern 80 and said second surgical inventory 10, 45, 50, 100, 200 represented by the second optical pattern 90 as seen from a surgeon wearing said surgeon worn device 5 and provides said visualization to said optical display device 75. It should be noted that the visualization may also be a mixed visualization of virtual surgical inventories 10, 45, 50, 100, 200 and real surgical inventories 10, 45, 50, 100, 200. The real surgical inventories may be represented as a real time displaying of imaged surgical inventories at the surgical site or may be provided by a direct view by the surgeon through a semi opaque display 75, which allows a view through to the surgical site and displaying of virtual surgical inventories at the same time. The image-processing device 30 may further have an augmenting means 38 for augmenting one or more surgical inventories 10, 45, 50, 100, 200 and an operating trajectory of a surgical inventory 16, 46 (not illustrated in FIG. 3) onto the virtual visualization of the surgical inventory 10, 45, 50, 100, 200. The augmenting may be based on a recognized position and orientation of the optical sub-patterns 80a, 90a of the respective optical pattern 80, 90 with respect to a position and viewing direction 71 of the surgeon wearing the surgeon worn device 5, so as to visualize the surgical inventory 10, 45, 50, 100, 200 and e.g., an operating trajectory of a surgical inventory 16, 46 relative to the surgical inventories 10, 45, 50, 100, 200 represented by the optical patterns 80, 90. It should be noted that the pattern recognition means 32, the position-determining means 34, the visualization means 36 and the augmenting means 38 of the image-processing device 30 may be provided as separate computational units, but may also be implemented partially or as a whole in an integral computational system. The image-processing device 30 may be provided within the surgeon worn device 5 or may be provided remote thereof.

FIG. 4 illustrates an exemplary embodiment with the components being relevant for the visualization and augmentation, which can be conducted by the image-processing device 30, which may be a computer or any other computational capacity. For this purpose, the system is provided with an image-processing device 30 having a pattern recognition means 32 and a visualization means 36, as illustrated in FIG. 4. Further, the image-processing device 30 may have an augmenting means 38. The visualization means 36 being adapted for a virtual visualization 19 of the orientation of the guiding body 15 with respect to a patient's anatomy 100, based on the unique radio projection 25 of the radio dense geometry 20, here of the third radio dense sub-geometry 23 with its fiducial markers 24, as illustrated in FIG. 4. The unique radio projection 27 of the third radio dense sub-geometry 23, in particular the pattern of the fiducial markers 24, allows a determination of the position and orientation of the guiding device. This allows a virtual visualization 19 of the orientation of the guiding body 15. The image-processing device 30 further has an augmenting means 38 for augmenting the guiding trajectory 16 onto the virtual visualization 19 of the orientation of the guiding body 15, so as to visualize a traveling path 46 of at least one of a surgical implant or a surgical tool 45 to be implanted. In order to simplify the orientation of the surgeon, the augmenting means 38 may augment a reproducible scale 39 along the augmented guiding trajectory 16. This scale 39 may give the surgeon an idea where an implant tip or tool tip will end when being inserted along the guiding trajectory 16. This scale may also support the surgeon in selecting the correct implant/tool length. In combination with an image recognition and anatomy identification, a suggestion may be made t the surgeon which tool or implant is recommended to be used. The augmenting means may also augment a geometry related to an implant to be implanted with respect to a patient's anatomy 100, based on the unique radio projection 25 of the radio dense geometry 20, in particular based on the unique radio projection 27 of the fiducial markers 24 of the third radio dense sub-geometry 23, as illustrated in FIG. 4.

FIG. 5 illustrates a complimentary match of a first and second radio dense sub-geometry 26, 27 as part of a radio dense geometry 60 of a complementary pattern 29 of radio projections of first and second sub-geometry.

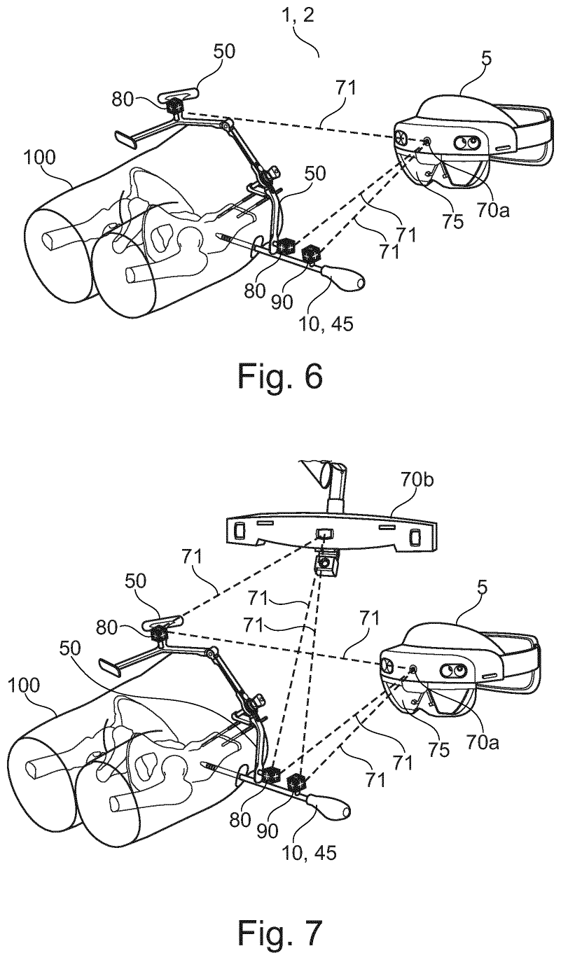

FIG. 6 illustrates the application of the surgical tracking system 2 to surgical inventories 10, 45, 50 with respect to an anatomy 100. The surgical tracking system operates as outlined above. The surgeon worn device 5 includes an imaging unit 70a, which images an optical pattern 80 on one side of a surgical reference body 50, a further optical pattern 80 on another side of the surgical reference body 50 in a respective viewing direction 71, and a further optical pattern 90 on a surgical instrument or tool 10, 45 in a respective further viewing direction 71. The two optical pattern 80 on the surgical reference body allow separately determining different segments of the surgical reference body 50 with respect to their position and orientation with respect to each other. The (not illustrated) image-processing device determines the position and orientation of the optical patterns 80, 90 with respect to each other and allows visualization of the objects or surgical inventories 10, 45, 50 being in a fixed in known spatial position to the respective optical pattern 80, 90. The display 75 within the surgeon worn device 5 allows the surgeon to have a real time view on the surgical situation, which may include a real view through a semi opaque display 75, a virtual illustration of surgical inventory 10, 45, 50, and an augmented reality including surgical inventories.

FIG. 7 illustrates a further exemplary embodiment, where a further optical imaging device 70b is provided, which here is positioned within the environment of a surgical procedure. The further imaging device 70b may also image the objects, which are also imaged by the imaging device 70a in the surgeon worn device 5. The pattern recognition means 32 may recognize the position and orientation of the optical patterns 80, 90 or the sub-patterns 80a, 90a of the respective optical pattern 80, 90 and the position and orientation of the optical pattern 80, 90 or the sub-patterns 80a, 90a of the optical patterns 80, 90 based on an image collected by the second optical imaging device 70b and a stored representation of the optical patterns 80, 90. The position-determining means 34 determines a relative position and orientation of the surgeon worn device 5 and the second optical imaging device 70b with respect to the first, surgeon worn optical imaging device 70a, and determines a relative position and orientation of the optical patterns 80, 90 with respect to a position and viewing direction 71 of the surgeon worn optical imaging device 70a based on an image taken from the other optical imaging device 70b, the determined relative position and orientation of the surgeon worn device 5 and the other optical imaging device 70b with respect to the surgeon worn optical imaging device 70a and a stored representation of the respective optical patterns 80, 90. This allows that in case where no respective optical pattern 80, 90 or sub-pattern 80a, 90a is recognizable upon optical imaging by one of the surgeon worn optical imaging device 70a and the other optical imaging device 70b, the position-determining means 34 determines a relative position and orientation of an optical pattern 80, 90 with respect to a position and viewing direction 71 of the surgeon worn optical imaging device 70a based on imaging by the other of the optical imaging device 70b. Thus, even if the surgeon obscures a view in a particular viewing direction 71, e.g., by his hands, this obscured view may be compensated by the optical imaging of the respective other optical imaging device 70b.

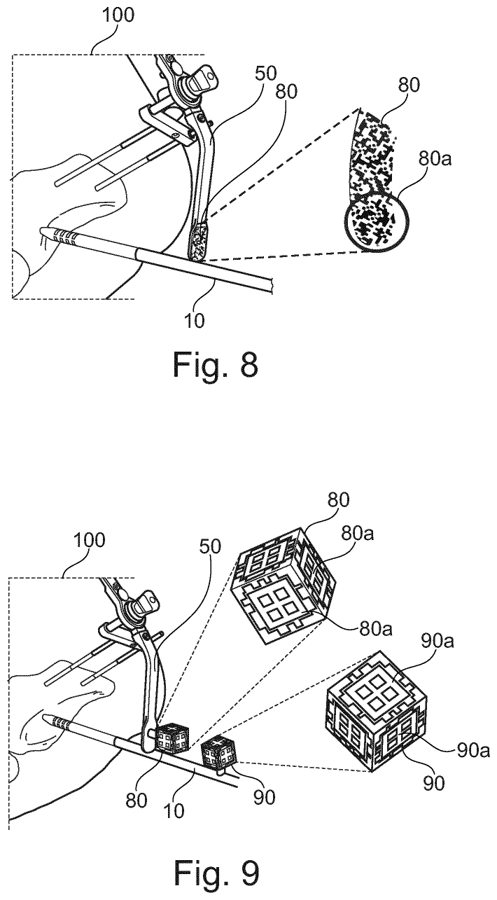

FIG. 8 illustrates a situation where an anatomy 100, in particular a bone thereof is represented with respect to the bone's position and orientation by a surgical reference body 50. The surgical reference body carries an optical pattern 80, which represents the surgical reference body 50 and thus the position and orientation of the bone. By imaging the optical pattern 80 or an optical sub pattern 80a thereof, it is possible to determine the position and orientation of the bone, without seeing the bone and without the need for a permanent or repeated fluoroscopic imaging of the bone. Thus, it is possible to operate a surgical instrument 10. The display 75 (not shown) on the surgeon worn device 5 may display the bone virtually and the surgical instrument 10 as overlapping real view.

An embodiment of the present disclosure can include a method for positioning a first surgical inventory such as tool 10 with reference to a second surgical inventory such as anatomy 100 using surgical guiding system 1 during a surgical procedure. Optical imaging S70 by first optical imaging device 70a on surgeon worn device 5 can be used to visualize a surgical environment including first optical pattern 80 representing a position and orientation of tool 10 and second optical pattern 90 representing a position and orientation of anatomy 100. A position and orientation of first sub-pattern 80a of the first optical pattern 80 and the position and orientation of a second sub-pattern 90a of the second optical pattern 90 can be determined by comparing an image collected by first optical imaging device 70a with a stored representation of the first optical pattern 80 and the second optical pattern 90. A relative position and orientation of the first optical pattern 80 and the second optical pattern 90 with respect to a position and viewing direction 71 of the first optical imaging device 70a based on the position and orientation of the recognized first sub-pattern 80a of the first optical pattern 80 and the position and orientation of the recognized second sub-pattern 90a of the second optical pattern 90 is then determined S34. Tool 10, represented by the first optical pattern 80, can now be placed and positioned with reference to anatomy 100, represented by the second optical pattern 90, by a surgeon based on visualizing S36 the tool and anatomy on optical display device 75 of surgeon worn device 5.

FIG. 9 illustrates that it is also possible to provide the instrument 10 with a representing optical pattern, as illustrated in FIG. 6 or 7, so that the entire situation can be virtually displayed. The optical pattern 80, 90 or sub patterns 80a, 90a, have a different design, but serve for a similar purpose. The different designs illustrated in FIG. 8 and FIG. 9 may have different advantages with respect to the side from which they are imaged and reveal the pattern information. Further, it is possible to augment an implant out of e.g., a database into the situation (not shown here) in order to give the surgeon an impression whether the selected implant fits the required purpose.

As describe above, the image-processing device 30 has a pattern recognition means 32 for recognizing the position and orientation of the at least sub-pattern 80a of the optical pattern 80 with respect to a position and viewing direction 71 of the imaging device 70 based on an image taken from the optical imaging device 70 and a stored representation of the optical pattern. Further, the image-processing device 30 has a visualization means 38 for virtually visualizing a surgical instrument 10 represented by the optical imaging device 70 and virtually visualizing a surgical reference body 50 represented by the optical pattern 80. Alternatively, the visualization means 36 virtually visualize a surgical instrument 10 represented by the optical pattern 80 and virtually visualize a surgical reference body 50 represented by the optical imaging device 70, depending onto which of the instrument 10 and the reference body 50, the imaging device 70 and the pattern 80 are mounted. If an augmenting means 38 is provided, the augmenting means may augment a moving axis or trajectory of an instrument, a scale or even a virtual instrument or a virtual implant to the visualization. The augmented items may be provided from a conversion process which converts at least two 2-dimensional images to a 3-dimensional image, or from virtually stored items from a data base. Further, additional information may be augmented, like quantitative scales, implant properties or identifiers etc., which may help the surgeon in identifying the correct measures and items, as illustrated e.g., in FIG. 4. The augmenting means may augment a predetermined operating trajectory 16, 46 of a surgical instrument 10 onto the virtual visualization of the surgical instrument 10 based on a recognized position and orientation of the at least sub-pattern 80a of the optical pattern 80 with respect to a position and viewing direction 71 of the imaging device 70, so as to visualize an operating path 46 of the surgical instrument 10 relative to a surgical reference body 50 represented by the optical pattern 80. This augmenting may take place on a screen or even in augmenting glasses worn by the surgeon during surgery.

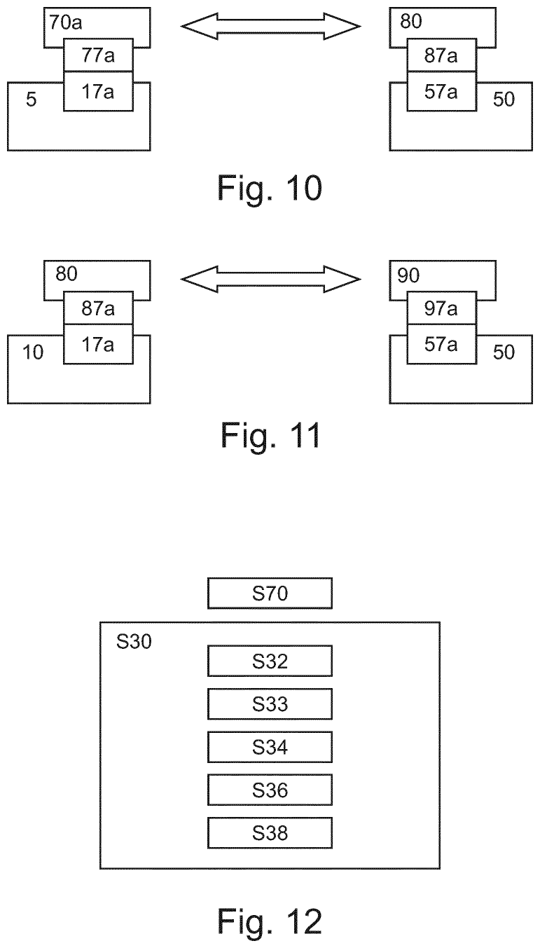

FIG. 10 illustrates that the optical imaging device 70a may have a mechanical interface 77a to be coupled to a positive fit receptacle 17a of the surgeon worn device 5 for forming a unit having a reproducible relation between a position of said surgeon worn device 5 and the position and viewing direction 71 of the optical imaging device 70a. Likewise, the optical pattern 80 may comprises a mechanical interface 87a to be coupled to a positive fit mechanical interface 57a of a surgical reference body 50 for forming a unit having a reproducible relation between a geometry of said surgical reference body 50, and the position and orientation of the optical pattern 80. Instead of providing releasable receptacles, it is also possible to connect fixedly the optical imaging device 70a to the surgeon worn device 5 for forming a unit having a reproducible relation between a surgeon worn device 5 and the position and viewing direction 71 of the optical imaging device 70a. Likewise, the optical pattern 80 may fixedly mounted to a surgical reference body 50 for forming a unit having a reproducible relation between a geometry of said surgical reference body 50, and the position and orientation of the optical pattern 80.

FIG. 11 illustrates that the first optical pattern 80 may have a mechanical interface 87a to be coupled to a positive fit receptacle 17a of a surgical inventory, e.g., an instrument 10 for forming a unit having a reproducible relation between a position of the first optical pattern 80 and the instrument 10. Likewise, the second optical pattern 90 may comprises a mechanical interface 97a to be coupled to a positive fit mechanical interface 57a of a further surgical inventory, e.g., a surgical reference body 50 for forming a unit having a reproducible relation between a geometry of said surgical reference body 50, and the position and orientation of the second optical pattern 90. Instead of providing releasable receptacles, it is also possible to connect fixedly the optical pattern 80 to e.g. an instrument 10 for forming a unit having a reproducible relation between the first optical pattern 80 and the instrument 10. Likewise, the second optical pattern 90 may fixedly mounted to a surgical reference body 50 for forming a unit having a reproducible relation between a geometry of said surgical reference body 50, and the position and orientation of the second optical pattern 90.

FIG. 12 illustrates a method for assisting positioning an application of implants/tools with respect to a patient's anatomy. The method includes processing imaging S30, which may include recognizing pattern(s) S32 and comparing recognized (sub-)pattern(s) with predetermined pattern(s) S33. Further, processing imaging S30 may include determining position and orientation of (sub-)pattern(s) S34, visualization S36 of the items and augmenting S38 e.g., a guiding trajectory, implant/tool/instrument contours or illustrations. For providing a relative position and orientation of an optical imaging device and an optical pattern, the method may include taking an optical image S70 of the pattern with the imaging device.

### REFERENCES

1 surgical guiding system for computer-assisted-surgery CAS
2 surgical tracking system
5 surgeon worn device, holo-lens, head mounted device
10 surgical guiding device/surgical instrument
11 proximal end of the surgical guiding device/surgical instrument
12 distal end of the surgical guiding device/surgical instrument
15 guiding body
15b guiding channel of guiding body
16 guiding trajectory/cutting plane trajectory of the guiding body/operating trajectory of surgical instrument
17 knob/handle of surgical guiding device
17a mechanical interface of surgical inventory, e.g., an instrument, for optical pattern
18 tip of surgical guiding device
19 virtual visualization of a guiding body/an orientation of the guiding body
20 radio dense geometry of surgical guiding device
21 first radio dense sub-geometry of surgical guiding device
22 second radio dense sub-geometry of surgical guiding device
23 third radio dense sub-geometry of surgical guiding device
25 radio projection of radio dense geometry of surgical guiding device
26 radio projection of the first sub-geometry of surgical guiding device
27 radio projection of the second sub-geometry of surgical guiding device
28 unique radio projection of the third sub-geometry of surgical guiding device
29 complementary pattern of radio projections of first/second sub-geometry
30 image-processing device
32 recognition means for pattern recognition
34 position-determining means for determining a position of recognized optical patterns
36 visualization means
38 augmenting means for augmenting the guiding trajectory
39 reproducible scale along augmented guiding trajectory
45 surgical implant/surgical tool
46 traveling path of a surgical implant/surgical tool to be inserted and guided/extended operating trajectory of surgical instrument
50 surgical reference body
51 first portion of surgical reference body
52 second portion of surgical reference body
57a mechanical interface of surgical inventory, e.g., a reference body, for optical pattern
60 radio dense geometry of surgical reference body
61 first radio dense sub-geometry of surgical reference body
62 second radio dense sub-geometry of surgical reference body
66 unique radio projection of the first sub-geometry of surgical reference body
67 unique radio projection of the second sub-geometry of surgical reference body
68 unique radio projection of the third sub-geometry of surgical reference body
70a first optical imaging device/on surgeon worn device/on head mounted device HMD
70b second optical imaging device in surgical environment remote from HMD
71 viewing direction of optical imaging device
75 optical display device/on surgeon worn device/on head mounted device HMD
77a mechanical interface of optical imaging device for surgeon worn device
80 first optical pattern
80a first (optical) sub-pattern
82 even raster of optical pattern
87a mechanical interface of first optical pattern to be connected to surgical inventory
90 second optical pattern
90a second (optical) sub-pattern
97a mechanical interface of second optical pattern to be connected to surgical inventory
100 patient's anatomy
200 Fluoroscopic imaging device
S30 processing imaging
S31 identifying coverage of (sub-)pattern(s)
S32 recognizing pattern(s)
S34 determining position and orientation of (sub-)pattern(s)/w.r.t. each other
S36 visualization
S38 augmenting for augmenting the guiding trajectory
S70 taking optical imaging

1. A surgical tracking system for tracking a plurality of surgical inventories with respect to each other, the surgical tracking system comprises:
a first optical pattern representing a position and orientation of a first surgical inventory, the first optical pattern having at least one first unique optical sub-pattern;
a second optical pattern representing a position and orientation of a second surgical inventory, the second optical pattern having at least one second unique optical sub-pattern;
a surgeon worn device comprising a first optical imaging device and an optical display device;
an image-processing device comprising
a pattern recognition means being adapted for recognizing the position and orientation of the first sub-pattern of the first optical pattern and the position and orientation of second sub-pattern of the second optical pattern based on an image collected by said first optical imaging device and a stored representation of the first optical pattern and the second optical pattern;
a position-determining means being adapted for determining a relative position and orientation of the first optical pattern and the second optical pattern with respect to a position and viewing direction of the first optical imaging device based on the position and orientation of the first sub-pattern of the first optical pattern and the position and orientation of second sub-pattern of the second optical pattern recognized by said pattern recognition means,
and
a visualization means being adapted for virtually visualizing said first surgical inventory represented by the first optical pattern and said second surgical inventory represented by the second optical pattern as seen from a surgeon wearing said surgeon worn device and providing said visualizing to said optical display device.

2. The surgical tracking system of claim 1, further comprising a second optical imaging device to be positioned within the environment of a surgical procedure,
wherein said pattern recognition means being adapted for recognizing the position and orientation of the first sub-pattern of the first optical pattern and the position and orientation of the second sub-pattern of the second optical pattern based on an image collected by said second optical imaging device and a stored representation of the first optical pattern and the second optical pattern;
wherein the position-determining means being adapted for determining a relative position and orientation of the surgeon worn device and the second optical imaging device with respect to the first optical imaging device, and for determining a relative position and orientation of the first optical pattern and the second optical pattern with respect to a position and viewing direction of the first optical imaging device based on an image taken from the second optical imaging device, the determined relative position and orientation of the surgeon worn device and the second optical imaging device with respect to the first optical imaging device and a stored representation of the first optical pattern and the second optical pattern.

3. The surgical tracking system of claim 2, wherein the image-processing device is adapted for identifying whether for at least one of the first optical pattern and the second optical pattern image no respective sub-pattern is recognizable upon optical imaging by one of the first optical imaging device and the second optical imaging device,
wherein the position-determining means being adapted for determining a relative position and orientation of the first optical pattern and the second optical pattern with respect to a position and viewing direction of the first optical imaging device based on imaging by the other of the first optical imaging device and the second optical imaging device.

4. The surgical tracking system of claim 1, wherein the image-processing device further comprises an augmenting means being adapted for augmenting at least one of a further surgical inventory and an operating trajectory of a surgical inventory onto the virtual visualization of the first surgical inventory and the second surgical inventory based on a recognized position and orientation of the first optical sub-pattern of the first optical pattern and the second optical sub-pattern of the second optical pattern with respect to a position and viewing direction of the first optical imaging device, so as to visualize said further surgical inventory and said operating trajectory of a surgical inventory, respectively, relative to said first surgical inventory represented by the first optical pattern and said second surgical inventory represented by the second optical pattern.

5. The surgical tracking system of claim 1, further comprising at least one of a first surgical inventory and a second surgical inventory, wherein the respective first optical pattern and second optical pattern is inseparable mounted to the corresponding respective one of the at least first surgical inventory and second surgical inventory, wherein the respective optical pattern representing a position and orientation of the respective surgical inventory.

6. The surgical tracking system of claim 1, wherein at least one of a first surgical inventory and a second surgical inventory is at least one of a surgical instrument, surgical implant, a surgical reference body, a patient's anatomy, and a fluoroscopic imaging device, wherein the at least one of the first surgical inventory and the second surgical inventory comprises a radio dense geometry having a unique radio projection for each proximal to distal orientation of the respective one of the first surgical inventory and the second surgical inventory, and being fixedly and spatially reproducibly connected to the respective one of the first surgical inventory and the second surgical inventory.

7. The surgical tracking system of claim 1, wherein at least one of the first optical pattern and the second optical pattern comprises a mechanical interface to be coupled to a positive fit receptacle of a surgical inventory for forming a unit having a reproducible positional relation between a geometry of said surgical inventory, and the respective optical pattern.

8. The surgical tracking system of claim 1, wherein the optical pattern is a three-dimensional optical pattern.

9. The surgical tracking system of claim 1, wherein at least one of the first optical pattern and the second optical pattern is composed of a geometrically even raster of light and dark fields, in particular a raster of squared light and dark fields, in particular a raster of light and black fields.

10. The surgical tracking system of claim 1, wherein at least one of the first optical pattern and the second optical pattern is composed of a geometrically even raster of fields of different colors, in particular a raster of squared colored fields, in particular a raster of color gradient fields.

11. The surgical tracking system of claim 5, wherein the surgical inventory is a surgical guiding device further comprising:
a guiding body having a longitudinal extension from a proximal end of the surgical guiding device to a distal end of the surgical guiding device, and being adapted for guiding at least one of a longitudinal surgical implant and a longitudinal tool, and having a guiding trajectory extending along the guiding body and succeeding in distal direction along a traveling path of at least one of a surgical implant and a surgical tool to be inserted and guided,
a radio dense geometry being located in a predetermined spatial position and orientation with respect to the guiding body, and being adapted for providing a unique radio projection for each proximal to distal orientation of the guiding body.

12. The surgical tracking system of claim 5, wherein the surgical inventory is a surgical reference body comprising:
a radio dense geometry being fixedly and spatially reproducibly connected to the surgical reference body,
a reference body portion having a mechanical interface for being connected to a patient's anatomy,
wherein the radio dense geometry has a unique radio projection for each proximal to distal orientation of the surgical reference body, so that the radio dense geometry allows determination of the spatial position and orientation of the surgical reference body based on a two dimensional radio projection of at least a part of the surgical reference body.

13. The surgical tracking system of claim 5, wherein the surgical reference body comprises:
a radio dense geometry having a first radio dense sub-geometry and a second radio dense sub-geometry each being fixedly and spatially reproducibly connected to the surgical reference body,
a first reference body portion having a mechanical interface for being connected to a patient's anatomy,
a second reference body portion having a mechanical interface for being connected to a patient's anatomy,
wherein each of the first radio dense sub-geometry and the second radio dense sub-geometry has a unique radio projection for each proximal to distal orientation of the surgical reference body, so that each of the first radio dense sub-geometry and the second radio dense sub-geometry alone allows determination of the spatial position and orientation of the surgical reference body based on a two dimensional radio projection of at least a part of the surgical reference body,
wherein the first radio dense sub-geometry is allocated to the first reference body portion, and the second radio dense sub-geometry is allocated to the second reference body portion.

14. A surgical tracking method for tracking a plurality of surgical inventories with respect to each other, the surgical tracking method comprises:
optical imaging by a first optical imaging device on a surgeon worn device a surgical environment including a first optical pattern representing a position and orientation of a first surgical inventory and a second optical pattern representing a position and orientation of a second surgical inventory,
recognizing a position and orientation of a first sub-pattern of the first optical pattern and the position and orientation of a second sub-pattern of the second optical pattern based on an image collected by said first optical imaging device and a stored representation of the first optical pattern and the second optical pattern;
determining a relative position and orientation of the first optical pattern and the second optical pattern with respect to a position and viewing direction of the first optical imaging device based on the position and orientation of the recognized first sub-pattern of the first optical pattern and the position and orientation of the recognized second sub-pattern of the second optical pattern;
visualizing said first surgical inventory represented by the first optical pattern and said second surgical inventory represented by the second optical pattern as seen from a surgeon; and
providing said visualizing to an optical display device on said surgeon worn device.

15. The surgical tracking method of claim 14, further comprising
optical imaging by a second optical imaging device positioned within the environment of a surgical procedure and apart from the first optical imaging device a surgical environment including a first optical pattern representing a position and orientation of a first surgical inventory and a second optical pattern representing a position and orientation of a second surgical inventory,
recognizing a position and orientation of a first sub-pattern of the first optical pattern and the position and orientation of a second sub-pattern of the second optical pattern based on an image collected by said second optical imaging device and a stored representation of the first optical pattern and the second optical pattern;
determining a relative position and orientation of the second optical imaging device with respect to the first optical imaging device;
determining a relative position and orientation of the first optical pattern and the second optical pattern with respect to a position and viewing direction of the first optical imaging device based on an image taken from the second optical imaging device, the determined relative position and orientation of the surgeon worn device and the second optical imaging device with respect to the first optical imaging device and a stored representation of the first optical pattern and the second optical pattern.

16. The surgical tracking method of claim 14, and further comprising
identifying whether for at least one of the first optical pattern and the second optical pattern image no respective sub-pattern is recognizable upon optical imaging by one of the first optical imaging device and the second optical imaging device,
determining a relative position and orientation of the first optical pattern and the second optical pattern with respect to a position and viewing direction of the first optical imaging device based on optical imaging by the other of the first optical imaging device and the second optical imaging device.

17. The surgical tracking method of claim 14, further comprising
augmenting at least one of a further surgical inventory and an operating trajectory of a surgical inventory onto the virtual visualization of the first surgical inventory and the second surgical inventory based on a recognized position and orientation of the first optical sub-pattern of the first optical pattern and the second optical sub-pattern of the second optical pattern with respect to a position and viewing direction of the first optical imaging device,
visualizing said further surgical inventory and said operating trajectory of a surgical inventory, respectively, relative to said first surgical inventory represented by the first optical pattern and said second surgical inventory represented by the second optical pattern.

18. The surgical tracking method of claim 14, further comprising
fluoroscopic imaging of a surgical environment including said first surgical inventory and said second surgical inventory, each having fixedly and spatially reproducibly connected thereto a radio dense marker having a unique radio projection for each proximal to distal orientation of said surgical inventory;
determining a position and orientation of each of said surgical inventories based on the fluoroscopic imaging and a stored spatial relation of each of the surgical inventories and a respective radio dense geometry with respect to each other;
establishing coincidence between a position and orientation of each of said surgical inventories determined based on the fluoroscopic imaging of respective radio dense markers and a position and orientation of each of said surgical inventories determined based on the optical imaging of respective optical markers.

19. A surgical navigation method for navigating a plurality of surgical inventories with respect to each other, the surgical navigation method comprises:
(A) fluoroscopic imaging of a surgical environment including said plurality of surgical inventories, each having fixedly and spatially reproducibly connected thereto a radio dense marker having a unique radio projection for each proximal to distal orientation of said surgical inventory;
(B) determining a position and orientation of each of said plurality of surgical inventories based on the fluoroscopic imaging and a stored spatial relation of each of the surgical inventories and a respective radio dense geometry with respect to each other;
(C) optical imaging by a first optical imaging device on a surgeon worn device a surgical environment including a first optical pattern representing a position and orientation of a first surgical inventory and a second optical pattern representing a position and orientation of a second surgical inventory,
(D) recognizing a position and orientation of a first sub-pattern of the first optical pattern and the position and orientation of a second sub-pattern of the second optical pattern based on an image collected by said first optical imaging device and a stored representation of the first optical pattern and the second optical pattern;
(E) determining a relative position and orientation of the first optical pattern and the second optical pattern with respect to a position and viewing direction of the first optical imaging device based on the position and orientation of the recognized first sub-pattern of the first optical pattern and the position and orientation of the recognized second sub-pattern of the second optical pattern;
(F) establishing coincidence between a position and orientation of each of said plurality of surgical inventories determined based on the fluoroscopic imaging of respective radio dense markers and a position and orientation of each of said plurality of surgical inventories determined based on the optical imaging of respective optical markers;
(G) visualizing said first surgical inventory represented by the first optical pattern and said second surgical inventory represented by the second optical pattern as seen from a surgeon;
(H) providing said visualizing to an optical display device on a surgeon worn device; and
(I) iteratively repeating steps (A), (B), (C), (D), (E), (G), and (H) for navigating a plurality of surgical inventories based on optical imaging.

20. The surgical tracking method of claim 14,
wherein recognizing a position and orientation of a first sub-pattern of the first optical pattern and the position and orientation of a second sub-pattern of the second optical pattern includes determining position and orientation of a first sub-pattern of the first optical pattern and the position and orientation of a second sub-pattern of the second optical pattern based on an image collected by said first optical imaging device and a stored representation of the first optical pattern and the second optical pattern;
wherein the method further comprises:
placing said first surgical inventory represented by the first optical pattern with reference to said second surgical inventory represented by the second optical pattern as seen from a surgeon based on visualizing said first surgical inventory and second surgical inventor on an optical display device of said surgeon worn device.
